(19) United States
(12) Patent Application Publication
(10) Pub. No.: US 2019/0274698 A1
(43) Pub. Date: Sep. 12, 2019

(54) UROLITH CAPTURE AND RETRIEVAL DEVICE FOR URINARY TRACT STONES

(71) Applicant: The Regents of the University of California, Oakland, CA (US)

(72) Inventors: Kamaijot Kaler, Orange, CA (US); Daniel Meller, Irvine, CA (US); Roshan M. Patel, Huntington Beach, CA (US); Ralph V. Clayman, Irvine, CA (US)

(73) Assignee: The Regents of the University of California, Oakland, CA (US)

(21) Appl. No.: 16/253,015

(22) Filed: Jan. 21, 2019

Related U.S. Application Data
(60) Provisional application No. 62/620,220, filed on Jan. 22, 2018.

(51) Int. Cl. A61B 17/221

(52) U.S. Cl. CPC A61B 17/221; A61B 2017/2212; A61B 2218/002

(57) ABSTRACT

Provided herein are devices related to the removal of uroliths, including kidney, ureteral and/or bladder stones. In one embodiment, the device comprises a mesh or sponge that is removably connected to a catheter, adapted for the removal of urolith fragments of the size 2 mm or less. In one embodiment, the device includes a catheter that is either solid or hollow, the latter for the irrigation of various types of fluid (either standard irrigation fluid (e.g. saline or water) or a material (e.g. blood, fibrin glue, etc.) that would form a clot like substance within the interstices of the device thereby increasing its ability to entrap fragments within the interstices of the basket or nearby.

### CROSS-REFERENCE TO RELATED APPLICATIONS

The present application claims the benefit of priority under 35 U.S.C. § 119(e) of provisional application Ser. No. 62/620,220, filed Jan. 22, 2018, the contents of which are hereby incorporated by reference.

### FIELD OF THE INVENTION

The present disclosure relates to devices related to urolith capture and retrieval.

### BACKGROUND OF THE DISCLOSURE

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Under current practice, uroliths (such as kidney, ureteral and/or bladder stones) are removed by breaking them into fragments using laser and/or shock wave lithotripsy and/or pneumatic lithotripsy. Current practices do not enable removal of stones less than 2 mm from the patient. It has long been assumed that these small stone fragments, which are unable to be removed by current “stone removal baskets”, would simply wash from the body by the action of gravity and/or urine flow. Recent literature shows that small stone fragments less than 2 millimeter (mm) are not completely washed from the body by these forces in as many as 50% of patients undergoing fragmentation therapy. These remaining stones may provide the nidus for recurrent and larger stone formation necessitating repeat surgical therapy or pain resulting in emergency room care. Thus there remains a need in the art for new devices and methods for effectively removing small urolith fragments in order to render patients truly stone free.

### SUMMARY OF THE DISCLOSURE

Various embodiments disclosed herein include a device comprising: a mesh and/or sponge removably connected to a catheter, wherein the device is capable of removing urolith fragments smaller than or equal to 2 millimeters. In one embodiment, the catheter is either solid or hollow. In one embodiment, a hollow catheter provides for the irrigation of a fluid. In one embodiment, the fluid is saline, water, blood, or fibrin glue, or combinations thereof. In one embodiment, the device is surgically insertable in the urinary tract of a patient in need thereof. In one embodiment, the urolith fragments comprise fragments of kidney, ureteral, and/or bladder stones. In one embodiment, the device removes urolith fragments after a urological procedure. In one embodiment, the urological procedure is percutaneous nephrolithotomy, ureteroscopy, cystoscopy, and/or cystolithopaxy combined with lithotripsy. In one embodiment, the mesh comprises interwoven monofilament. In one embodiment, the interwoven monofilament comprises nylon and/or fluorocarbon.

Various embodiments disclosed herein also include a method of removing uroliths and fragments thereof in a patient, the method comprising: providing a device comprising a mesh and/or sponge removably connected to a catheter; and removing uroliths and fragments thereof by surgically inserting the device in the patient around the location of the uroliths. In one embodiment, the urolith fragments comprise fragments of kidney, ureteral, and/or bladder stones. In one embodiment, the device removes urolith fragments after a urological procedure. In one embodiment, the device is capable of removing urolith fragments smaller than or equal to 2 millimeters. In one embodiment, the urological procedure is percutaneous nephrolithotomy, ureteroscopy, cystoscopy, and/or cystolithopaxy combined with lithotripsy. In one embodiment, the mesh comprises interwoven monofilament. In one embodiment, the interwoven monofilament comprises nylon and/or fluorocarbon.

Other embodiments disclosed herein include a method of treating a disease comprising: providing a device comprising a mesh and/or sponge removably connected to a catheter; and treating the disease by removing uroliths and fragments thereof by surgically inserting the device in a patient around the location of the uroliths. In one embodiment, the catheter is either solid or hollow. In one embodiment, a hollow catheter provides for the irrigation of a fluid. In one embodiment, the fluid is saline, water, blood, or fibrin glue, or combinations thereof. In one embodiment, the disease is an urological disease. In one embodiment, the urolith fragments comprise fragments of kidney, ureteral, and/or bladder stones. In one embodiment, the device is capable of removing urolith fragments smaller than or equal to 2 mm. In one embodiment, removal of the small urolith fragments less than 2 mm results in a longer period of being symptom-free for patients post-surgery, increases their quality of life, decreases the likely need for future medical procedures, and/or provides less financial impact on the health care system. In one embodiment, removal of the small urolith fragments less than 2 mm eliminates recurrent stone episodes and/or lengthens stone free periods for the patients.

Other features and advantages of the invention will become apparent from the following detailed description, taken in conjunction with the accompanying drawings, which illustrate, by way of example, various embodiments of the invention.

### DESCRIPTION OF THE DRAWINGS

Exemplary embodiments are illustrated in referenced figures. It is intended that the embodiments and figures disclosed herein are to be considered illustrative rather than restrictive.

FIG. 1 depicts, in accordance with embodiments herein, one embodiment of the urolith capture and retrieval device disclosed herein. The drawing depicts various views of the urolith capture and retrieval device.

FIG. 2 depicts, in accordance with embodiments herein, one embodiment of device disclosed herein illustrating different kinds of sponges. FIG. 2(A) depicts an example of a catheter; FIG. 2(B) depicts an example of a sponge; FIG. 2(C) depicts an example of a sponge.

FIG. 3 depicts, in accordance with embodiments herein, another embodiment of the device disclosed herein illustrating the different parts. FIG. 3(A) depicts a side perspective of the device looking at features inside the device; FIG. 3(B) depicts a top-down perspective of the device; FIG. 3(C) depicts a side perspective of the device looking at features from the outside.

FIG. 4 depicts, in accordance with embodiments herein, another embodiment of the device illustrating how the fragments would enter the basket and become entrapped. FIG. 4(A) depicts the fragment motion at position 1; FIG. 4(B) depicts the fragment motion at position 2; FIG. 4(C) depicts the fragment motion at position 3; FIG. 4(D) depicts the fragment motion at position 4; FIG. 4(E) depicts the fragment motion at position 5; FIG. 4(F) depicts the fragment motion at position 6.

### DETAILED DESCRIPTION

All references, publications, and patents cited herein are incorporated by reference in their entirety as though they are fully set forth. Unless defined otherwise, technical and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

As used herein, the term “fragments of uroliths” includes fragments of kidney, ureteral and/or bladder stones.

As described herein, in accordance with the various embodiments herein, the inventors have developed a device that may remove fragments of uroliths (kidney, ureteral and/or bladder stones), including fragments of less than 2 mm in size in the urinary tract, for example. Common current practice is to break large uroliths (urinary tract stones) into fragments using laser and/or shock wave lithotripsy and/or pneumatic lithotripsy. Fragments of stones greater than 2 mm are generally then removed with a with “baskets” or flushed from the urinary tract with saline irrigation. Current practices and technology do not enable removal of stones less than 2 mm from the patient. Until now, limited attention has been paid to the removal of <2 mm stone fragments, as these were assumed to be clinically insignificant. However, recent literature shows that residual fragments even those less than 2 mm result in more frequent stone related events including stone growth, need for repeat procedures and increased morbidity. In one embodiment, the device disclosed herein would fill a niche for removal of these very small stones (<2 mm).

In one embodiment, the present disclosure provides a device comprising a mesh and/or sponge removably connected to a catheter, wherein the device is capable of removing urolith fragments smaller than or equal to 2 millimeters. In one embodiment, the device is surgically insertable in the urinary tract of a patient in need thereof. In one embodiment, the urolith fragments comprise fragments of kidney, ureteral, and/or bladder stones. In one embodiment, the device removes urolith fragments after a urological procedure. In one embodiment, the urological procedure is percutaneous nephrolithotomy. In one embodiment, the mesh comprises interwoven monofilament. In one embodiment, the interwoven monofilament comprises nylon and/or fluorocarbon.

In one embodiment, surgical utilization and incorporation of the of the urolith capture and retrieval device disclosed herein may increase the total percentage of stone removal by capturing these very small stones less than 2 mm in length. In one embodiment, this may help to prevent and/or delay the return of the patient's symptoms. In another embodiment, this may increase the patient's quality of life, reduce the need for multiple future procedures, and is likely to result in less financial burden on the healthcare system.

In one embodiment, disclosed herein is a method of removing uroliths and fragments thereof in a patient, the method comprising, providing a device comprising a mesh and/or sponge removably connected to a catheter, and removing uroliths and fragments thereof by surgically inserting the device in the patient around the location of the uroliths. In one embodiment, the catheter is either solid or hollow, the latter for the irrigation of various types of fluid (either standard irrigation fluid (e.g. saline or water) or a material (e.g. blood, fibrin glue, etc.) that would form a clot like substance within the interstices of the device thereby increasing its ability to entrap fragments within the interstices of the basket or nearby. In one embodiment, the urolith fragments comprise fragments of kidney, ureteral, and/or bladder stones. In one embodiment, the device removes urolith fragments after a urological procedure. In one embodiment, the device is capable of removing urolith fragments smaller than or equal to 2 millimeters. In one embodiment, the urological procedure is percutaneous nephrolithotomy. In one embodiment, the mesh comprises interwoven monofilament. In one embodiment, the interwoven monofilament comprises nylon and/or fluorocarbon.

In one embodiment, the inventors have found that there are no devices currently available for removing very small stones less than or equal to 2 mm fragments from the kidney, ureter, and bladder after lithotripsy. An advantage of the device disclosed herein is that it enables the removal of these very small fragments, with the likely result of a longer period of being symptom-free for patients who are post-surgery, increasing their quality of life, decreasing the likely need for future medical procedures, and less financial impact on the health care system. More complete stone removal, as possible with this device, can eliminate recurrent stone episodes and/or lengthen stone-free (pain-free) periods for the patient by years. While current basket devices grasp individual or small numbers of uroliths (larger than 2 mm), there is no device that simultaneous captures many stones (and those smaller than 2 mm) by enmeshing them within a textured (sponge or otherwise) and/or woven surface. This novel mechanism is likely to increase the speed of endoscopic urolith removal, reducing operating time, expense, and risk. There is no device currently available that is capable of expanding and enmeshing stones 360° radially from its placement with a tubular organ, such as the ureter. Such innovation will improve efficiency and completeness of the urolithotomy procedure. The device itself is an inexpensive component of the total urolithotomy and/or nephrolithotomy and/or ureterolithotomy and/or cystolithotomy procedure, yet its use may significantly reduce future medical and surgical expenses for the patient over the ensuing years. In one embodiment, the device disclosed herein functions with an overcatheter that closes onto the device similar to current stone basket technology.

In one embodiment, disclosed herein is a method of treating a disease by providing a device comprising a mesh and/or sponge removably connected to a catheter; and treating the disease by removing uroliths and fragments thereof by surgically inserting the device in a patient around the location of the uroliths. In one embodiment, the catheter is either solid or hollow, the latter for the irrigation of various types of fluid (either standard irrigation fluid (e.g. saline or water) or a material (e.g. blood, fibrin glue, etc.) that would form a clot like substance within the interstices of the device thereby increasing its ability to entrap fragments within the interstices of the basket or nearby. In one embodiment, the disease is an urological disease. In one embodiment, the urolith fragments comprise fragments of kidney, ureteral, and/or bladder stones. In one embodiment, the device is capable of removing urolith fragments smaller than or equal to 2 mm. In one embodiment, removal of the small urolith fragments less than 2 mm results in a longer period of being symptom-free for patients post-surgery, increases their quality of life, decreases the likely need for future medical procedures, and/or provides less financial impact on the health care system. In one embodiment, removal of the small urolith fragments less than 2 mm eliminates recurrent stone episodes and/or lengthen stone free periods for the patients.

Embodiments of the present disclosure are further described in the following examples. The examples are merely illustrative and do not in any way limit the scope of the invention as claimed.

### EXAMPLES

### Example 1

### Urolith Capture and Retrieval Device

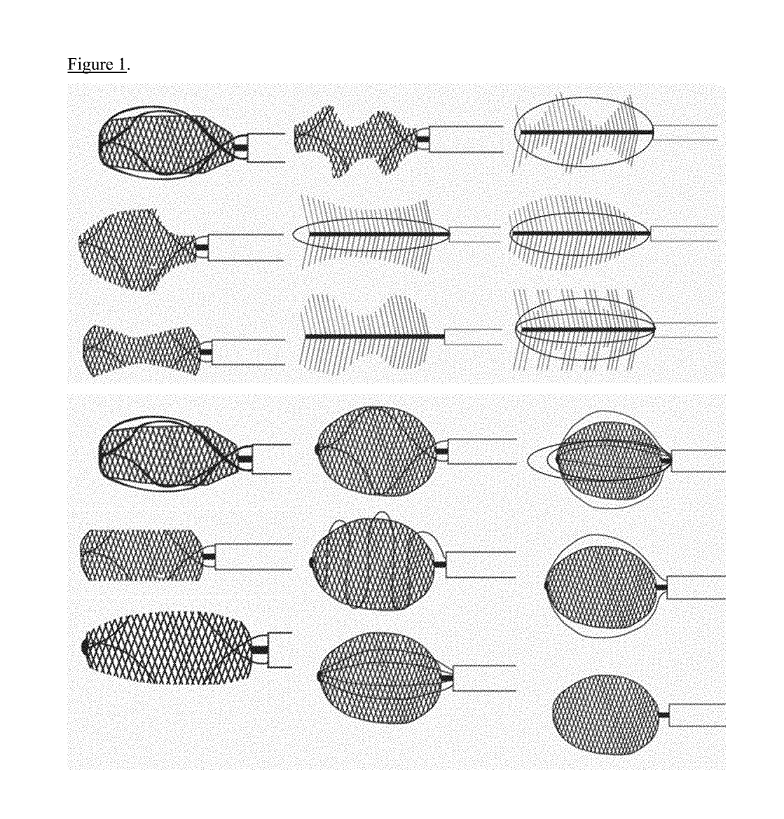

In one embodiment, a urolith capture and retrieval device for urinary tract stones as disclosed herein has a novel mesh and/or sponge type design that allows even the smallest uroliths (or, stones) to be captured and subsequently removed. The mesh device is made from interwoven monofilament (nylon, fluorocarbon) while the sponge device is made of a sponge-like material and can either be back or front-loaded through the endoscope. Further sponge material could be incorporated onto the monofilament as part of a modification during the initial production process or used separately as a sponge as further described herein and in the figures herein. For example, a “Capture and Retrieval device” could be used in the form of catheters as used with baskets up to 3 French or part of percutaneous stone removal device such as the Perc-N-Circle (Cook Medical). FIG. 1 discloses several prototypes of the device disclosed herein.

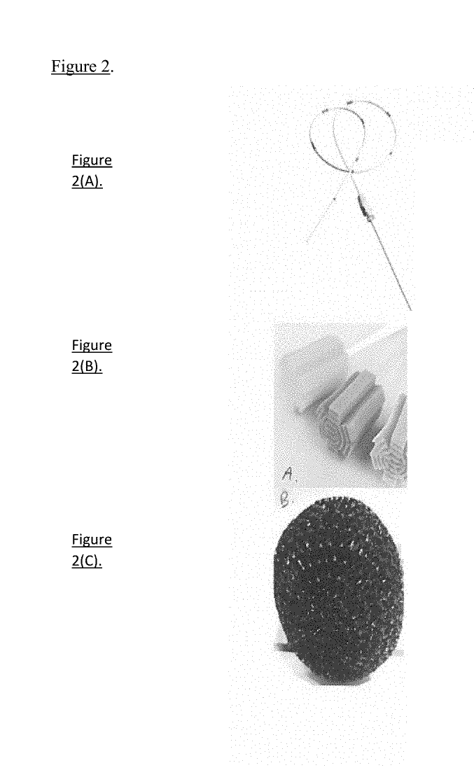

FIG. 2 illustrates a 3 French (P) ureteral catheter that can be used in conjunction with and connected to the urolith capture and retrieval device disclosed herein. In one embodiment, the catheter is used to irrigate, either standard irrigation fluid (e.g. saline or water), or a material (e.g. blood, fibrin glue, etc.), that would form a clot like substance within the interstices of the device thereby increasing its ability to entrap fragments within the interstices of the basket or nearby) and suction through it. In one embodiment, a sponge is attached or screw mounted to a 3F catheter, or for example, back loaded such as to a biopsy device (Cook Medical). In accordance with various embodiments herein, two design appearances of the sponge are illustrated in FIG. 2 herein: typical sponge material and collapsing sponge.

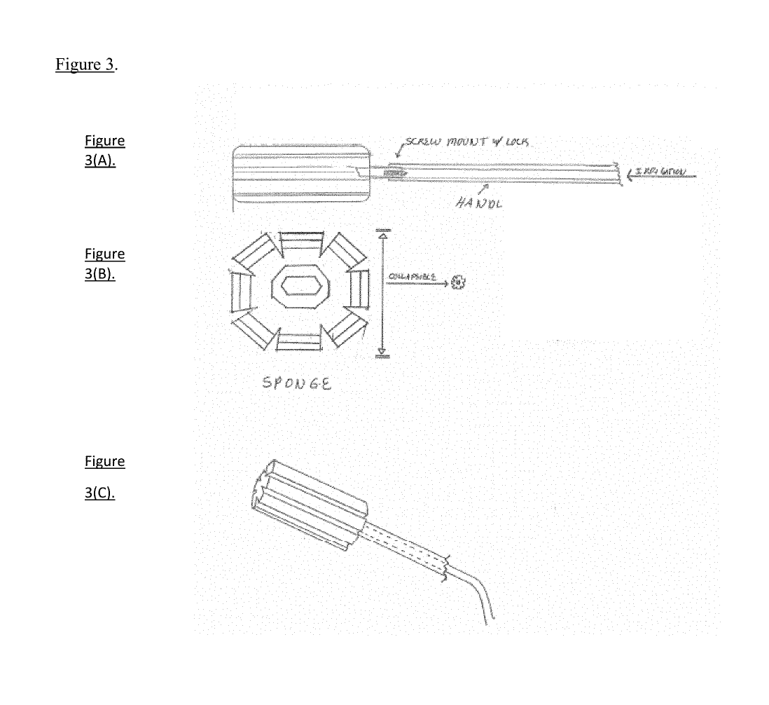

FIG. 3 provides another embodiment of the sponge stone retrieval device. In this embodiment, the device is screw mounted with lock to a ureteral catheter that is capable of providing irrigation (either standard irrigation fluid (e.g. saline or water) or a material (e.g. blood, fibrin glue, etc.) that would form a clot like substance within the interstices of the device thereby increasing its ability to entrap fragments within the interstices of the basket or nearby) and suction through the catheter. In one embodiment, 2F or 3F catheters of length 45-150 cm are taken, so that it can fit though a nephroscope/cystoscope or an ureteroscope, respectively.

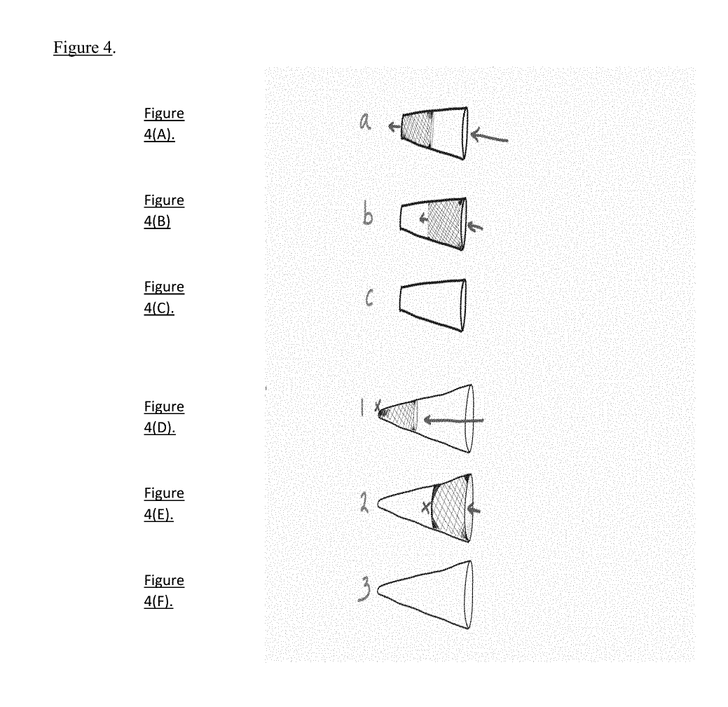

FIG. 4 provides another embodiment of the mesh basket, showing the mesh only on the top of the basket such that the bottom half is open, similar to a windsock so it can be dragged through fragments and have them entrapped in the upper half of the mesh. In one embodiment, FIG. 4 illustrates how the fragments would enter the basket to become entrapped.

In another embodiment, provided herein is a basket that is mounted on 2 wires—the standard one at the bottom of the basket that is used to push the basket forward and thereby allow it to open once it has cleared the sheath and a second wire attached to the top of the basket which also could be used to advance the basket out of its sheath. Once deployed, the topmost wire can be pulled back while the bottom wire is kept stationery—this in effect would open the mesh of the basket similar to the old child's toy often call a Chinese finger trap to a larger size such that when dragged into stone debris fragments up to 2 mm would enter the basket and then by keeping the topmost wire stationery, and pulling the bottommost wire caudal, the mesh is extended and the hole size reduced to under 1 mm.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps, some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof. Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a,” “an,” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A device comprising:
a mesh and/or sponge removably connected to a catheter,
wherein the device is adapted for removal for urolith fragments smaller than or equal to 2 millimeters.

2. The device of claim 1, wherein the catheter is either solid or hollow.

3. The device of claim 1, wherein a hollow catheter provides for the irrigation of a fluid.

4. The device of claim 3, wherein the fluid is saline, water, blood, or fibrin glue, or combinations thereof.

5. The device of claim 1, wherein the device is surgically insertable in the urinary tract of a patient in need thereof.

6. The device of claim 1, wherein urolith fragments comprises fragments of kidney, ureteral, and/or bladder stones.

7. The device of claim 1, wherein the device removes urolith fragments after a urological procedure.

8. The device of claim 7, wherein the urological procedure is percutaneous nephrolithotomy, ureteroscopy, cystoscopy, and/or cystolithopaxy combined with lithotripsy.

9. The device of claim 1, wherein the mesh comprises interwoven monofilament.

10. The device of claim 1, wherein the interwoven monofilament comprises nylon and/or fluorocarbon.

11. A method of removing uroliths and fragments thereof in a patient, comprising:
providing a device comprising a mesh and/or sponge removably connected to a catheter; and
removing a urolith and fragments thereof by surgically inserting the device in the patient.

12. The method of claim 11, wherein urolith fragments comprises fragments of kidney, ureteral, and/or bladder stones.

13. The method of claim 11, wherein the device is capable of removing urolith fragments smaller than or equal to 2 millimeters.

14. The method of claim 11, wherein the device removes urolith fragments after a urological procedure.

15. The method of claim 14, wherein the urological procedure is percutaneous nephrolithotomy, ureteroscopy, cystoscopy, and/or cystolithopaxy combined with lithotripsy.

16. The method of claim 11, wherein the mesh comprises interwoven monofilament.

17. The method of claim 11, wherein the interwoven monofilament comprises nylon and/or fluorocarbon.

18. A method of treating a disease comprising:
providing a device comprising a mesh and/or sponge removably connected to a catheter,
treating the disease by surgically inserting the device in a patient to remove a urolith or fragments thereof.

19. The method of claim 18, wherein the catheter is either solid or hollow.

20. The method of claim 18, wherein a hollow catheter provides for the irrigation of a fluid.

21. The method of claim 20, wherein the fluid is saline, water, blood, or fibrin glue, or combinations thereof.

22. The method of claim 18, wherein the disease is an urological disease.

23. The method of claim 18, wherein the urolith comprises fragments of kidney, ureteral, and/or bladder stones.

24. The method of claim 18, wherein the device is capable of removing a urolith fragment smaller than or equal to 2 mm.

25. The method of claim 18, wherein removal of the urolith results in a longer period of being symptom-free for patients post-surgery, increases their quality of life, decreases the likely need for future medical procedures, and/or provides less financial impact on the health care system.

26. The method of claim 18, wherein removal of the urolith eliminates recurrent stone episodes and/or lengthen stone free periods for the patients.
